(19) United States
(12) Patent Application Publication
(10) Pub. No.: US 2016/0242800 A1
(43) Pub. Date: Aug. 25, 2016

(54) ARTICULATING SURGICAL HAND INSTRUMENT

(71) Applicant: Cook Medical Technologies LLC, Bloomington, IN (US)

(72) Inventors: Jeffry Scott Melsheimer, Springville, IN (US); Brian Feng, Bloomington, IN (US); Amro Kamel, Bloomington, IN (US)

(21) Appl. No.: 15/017,921

(22) Filed: Feb. 8, 2016

Related U.S. Application Data
(60) Provisional application No. 62/120,487, filed on Feb. 25, 2015.

(51) Int. Cl. A61B 17/29; A61B 17/3201; A61B 17/295

(52) U.S. Cl. CPC A61B 17/2909; A61B 17/295; A61B 17/3201; A61B 2017/2923; A61B 2017/2924; A61B 2017/2943; A61B 2017/320044

(57) ABSTRACT

A surgical hand instrument may be inserted through a trocar to facilitate laparoscopic surgery. An end effector, such as a clamp, a spreader or scissors, is actuated by moving a pair of handles between a spread configuration and a contracted configuration. The end effector may be articulated relative to an elongate hollow shaft extending through the trocar with a steering wheel positioned between the pair of handles and operably coupled to the end effector by left and right turn cables and a pair of meshed gear sections.

### TECHNICAL FIELD

The present disclosure relates generally to articulating surgical hand instruments, and more particularly to an articulating surgical hand instrument suitable for use in laparoscopic surgery.

### BACKGROUND

Sometimes when performing a laparoscopic procedure, the surgeon will need to get access to an internal organ/tissue, manipulate it into a given position, and hold it there while surgery is taking place. This can sometimes be extremely difficult to impossible with currently available surgical hand instruments. While access to an internal organ/tissue may be accomplished by adding an alternative trocar access location to the patient, this option is often less than desirable, and sometimes not a realistic option.

The present disclosure is directed toward overcoming one or more of the problems set forth above.

### SUMMARY OF THE DISCLOSURE

In one aspect, a surgical hand instrument includes a pair of handles attached to a hub. The pair of handles includes a left handle pivotable relative to a right handle between a spread configuration and a contracted configuration. An elongate hollow shaft is attached to the hub. An end effector is movable between a first configuration and a second configuration. An articulation segment is attached at opposite ends to the elongate hollow shaft and the end effector, respectively. An articulation actuator is positioned between the pair of handles, and operably connected to the articulation segment by a left turn cable and a right turn cable. The end effector is operably coupled to the pair of handles by an effector cable, such that the end effector moves between the first and second configurations responsive to the pair of handles moving between the spread and contracted configurations. The end effector has a cross sectional dimension equal to, or less than, an outer diameter of the elongate hollow shaft in the second configuration, but the cross sectional dimension is greater than the outer diameter of the elongate shaft in the first configuration. A pair of meshed gear sections of the articulation segment rotates with respect to each other to articulate the end effector relative to the elongate hollow shaft responsive to movement of the articulation actuator.

In another aspect, a surgical instrument assembly includes the surgical hand instrument extending through a trocar.

In still another aspect, a method of operating a surgical hand instrument includes moving an end effector through a trocar while the end effector is in a second configuration. The surgical hand instrument is rotated about a trocar axis. The end effector is articulated relative to an elongate hollow shaft responsive to movement of an articulation actuator, a left turn cable and a right turn cable. The end effector is moved from a second configuration to a first configuration responsive to pivoting a left handle relative to a right handle, and moving an end effector cable. The surgical hand instrument is repositioned relative to the trocar. The end effector is moved from the first configuration to the second configuration. The step of articulating the end effector includes rotating a pair of meshed gear sections of an articulation segment relative to each other.

### BRIEF DESCRIPTION OF THE DRAWINGS

FIG. 1 is a side view of a surgical instrument assembly according to the present disclosure;

FIG. 2 is a side view of the surgical instrument assembly of FIG. 1, except showing the end effector articulated at an angle;

FIG. 3 is a side view of the surgical instrument assembly of FIG. 1, except showing the end effector in the second configuration;

FIG. 4 is a partial side schematic view of the surgical hand instrument according to the present disclosure;

FIG. 5 is a perspective view of the steering wheel articulation actuator with the turn cable mountings;

FIG. 6 is a side schematic view of the articulation segment and adjacent portions of a surgical hand instrument according to the present disclosure;

FIG. 7 is a partial perspective isometric view of surgical hand instrument, with the handle in a spread configuration;

FIG. 8 is a view similar to FIG. 6 except showing the handle in the contracted configuration;

FIG. 9 is a partial side view of a surgical hand instrument in which the end effector is a pair of scissors; and

FIG. 10 is a partial side view of still another surgical hand instrument in which the end effector is a spreader.

### DETAILED DESCRIPTION

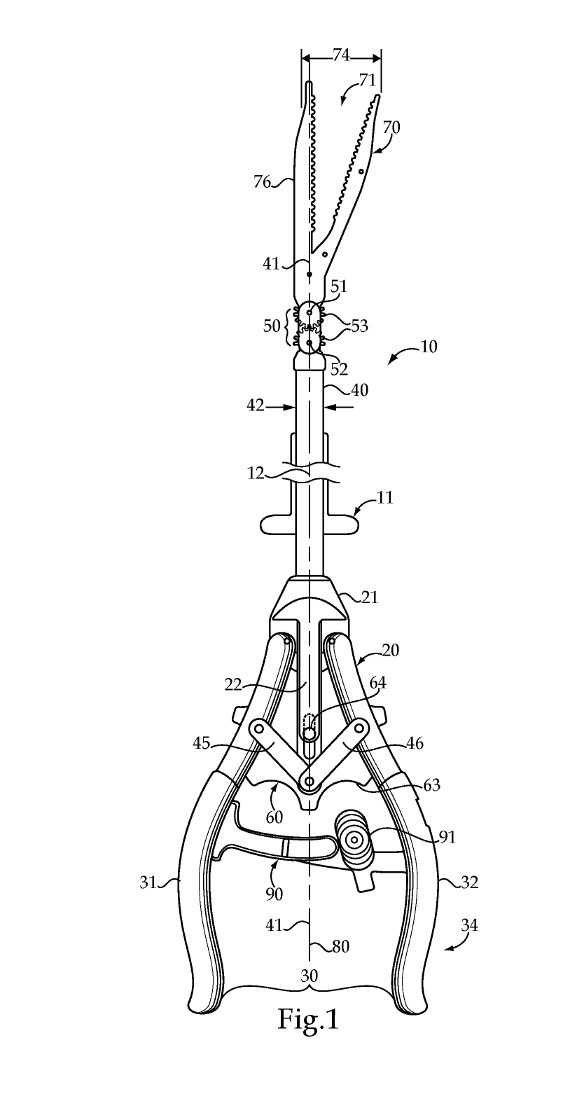

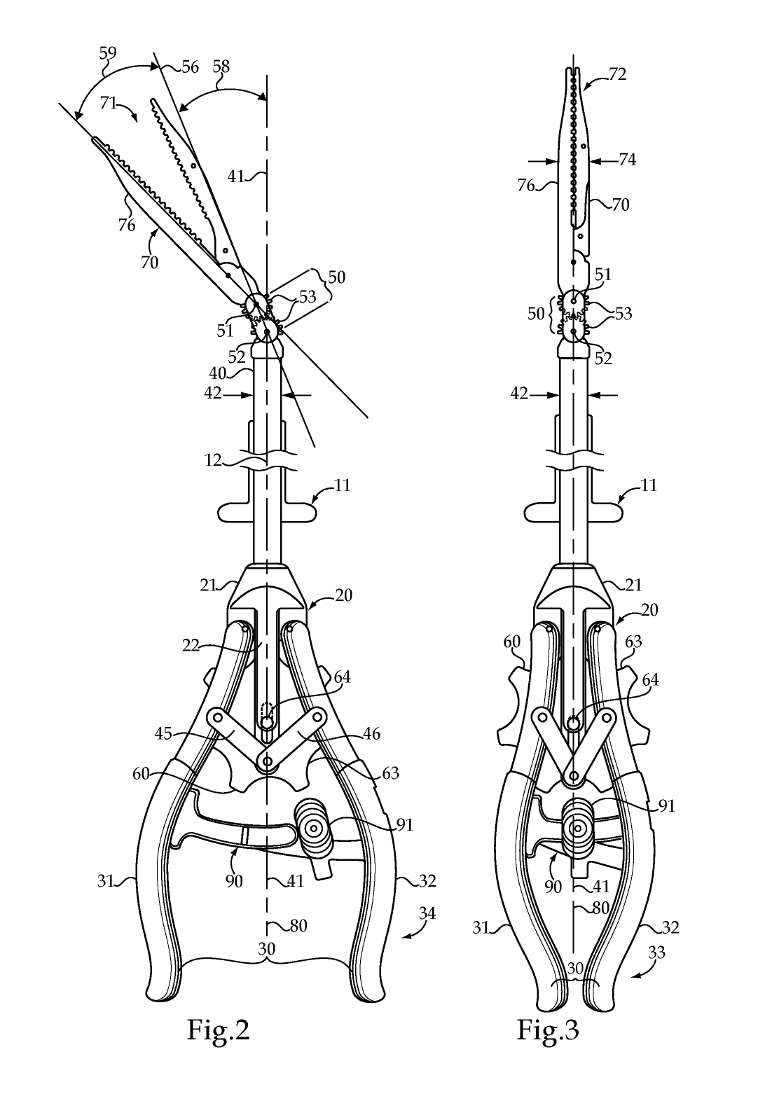

Referring initially to FIGS. 1-3, a surgical instrument assembly 10 is shown with a surgical hand instrument 20 extending through a trocar 11. Trocar 11 could be any suitable trocar available that includes a central cylindrical passage that extends along a trocar axis 12 for passing surgical hand instruments, laparoscopes and the like into a patient body cavity (not shown). Surgical hand instrument 20 includes a pair of handles 30 pivotally attached to a hub 21. The pair of handles 30 includes a left handle 31 and a right handle 32 that are pivotable relative to each other between a spread configuration 34 (FIGS. 1-2) and a contracted configuration 33 as shown in FIG. 3. The surgical hand instrument 20 includes an end effector 70 separated from an elongated hollow shaft 40 by an articulation segment 50. The opposite end of elongate hollow shaft 40 is attached to hub 21. Articulation segment 50 is attached at opposite ends 51, 52 to the elongate hollow shaft 40 and the end effector 70, respectively. End effector 70, which is shown as a clamp 76, is movable between a first configuration 71 as shown in FIGS. 1 and 2, and a second configuration 72 as shown in FIG. 3. An articulation actuator 60 is positioned between the pair of handles 30 and operably connected to the articulation segment 50 by left and right turn cables that are discussed infra. The end effector 70 is operably coupled to the pair of handles 30 by an effector cable (discussed infra) such that the end effector 70 moves between the first and second configurations 71, 72 responsive to the pair of handles 30 moving between the spread and contracted configurations 34, 33. The end effector 70 has a cross sectional dimension 74 that is equal to, or less than, an outer diameter 42 of the elongate hollow shaft 40 in the second configuration 72 (FIG. 3). The cross sectional dimension 74 is greater than the outer diameter 42 of the elongate hollow shaft 40 in the first configuration 71 (FIGS. 1 and 2). A pair of meshed gear sections 53 of the articulation segment 50 rotate with respect to each other to articulate the end effector 70 relative to the elongate hollow shaft 40 responsive to movement of the articulation actuator 60. As used in this disclosure, articulate and articulation mean a flexible joint between an end effector and an elongate hollow shaft.

Although surgical hand instrument 20 could be constructed to have a pistol grip handle configuration typical in some laparoscopic instruments, the illustrated embodiment has a structure where the left handle 31 and the right handle 32 are located on opposite sides of a plane 80 that contains centerline 41 of the elongate hollow shaft 40. Those skilled in the art will appreciate that other handle configurations relative to the elongate shaft 40 of the surgical hand instrument 20 would also fall within the intended scope of the present disclosure.

In the illustrated embodiment of the surgical hand instrument 20, the articulation actuator 60 takes the form of a steering wheel 63 that is rotatable about a steering axis 64. Nevertheless, those skilled in the art will appreciate that structures other than a steering wheel 63 could be utilized in order to actuate the articulation feature of the surgical hand instrument 20. In the illustrated embodiment, steering axis 64 is intersected by, and perpendicular to, the centerline 41 of hollow shaft 40. Nevertheless, those skilled in the art appreciate that the orientation of steering axis 64 could be different without departing from the intended scope of the present disclosure.

Although not necessary, surgical hand instrument 20 may include a ratchet 90 that is operably coupled to lock the pair of handles to prevent the handles from pivoting toward the spread configuration 34. This feature might be more desirable depending upon a chosen end effector 70, with the ratchet 90 being particularly useful when end effector 70 takes the form of a clamp 76. A release 91 may be operably coupled to disengage the ratchet 90 to permit the pair of handles to pivot toward the spread configuration 34. In the present illustration, ratchet 90 takes the form of two separate toothed extensions extending from the respective left handle 31 and right handle 32. The extensions include opposing teeth that interact and catch on one another in a manner well known in the art. Release 91 may simply operate by flexing the extensions slightly apart to facilitate spreading of the pair of handles 30. Nevertheless, those skilled in the art will appreciate that, if included, ratchet 90, and possibly release 91 could take on other forms known in the art with regard to other surgical hand instruments.

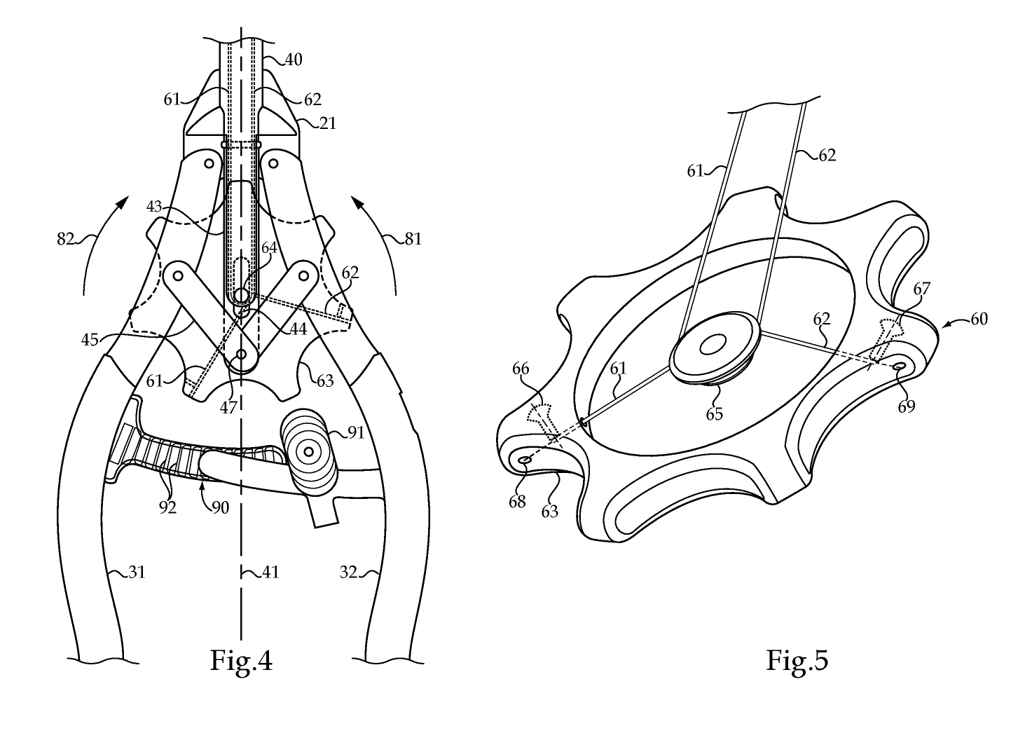

Referring now in addition to FIGS. 4-8, articulation actuator 60, which in the illustrated embodiment is a steering wheel 63, is operably coupled to articulate an effector 70 by left turn cable 61 and a right turn cable 62. One end of left turn table 61 is fixed to end effector 70 and extends around the left hand side of articulation segment 50, wraps counterclockwise around capstan 65 (FIG. 5) then to the left side of steering axis 64, and terminates at its opposite end at a mounting in steering wheel 63. During assembly, the terminal end of left turn cable 61 may extend out of through hole 68 in steering wheel 63. Proper tension may be set and held with a set screw 66, and the excess cable beyond through hole 68 may be cut off. In this way, when steering wheel 63 is rotated in a counter clockwise direction 81, left turn cable 61 increases in tension and this forces the first gear section 54 of the pair of meshed gear sections 53 to rotate in a counterclockwise direction 81 on second gear section 55. In a mirrored fashion, the right hand turned cable 62 has one end affixed to end effector 70, has a middle section that extends around the right hand side of articulation segment 50, down through elongate hollow shaft 40, wraps clockwise around capstan 65, then to the right hand side of steering axis 64 to a mounting location in steering wheel 63. During assembly, excess cable may extend out of through hole 69 while proper tensioning is set with set screw 67. The excess cable may then be cut off and discarded. Thus, when steering wheel 63 is rotated in a clockwise direction 82, tension in right turn table 62 is increased, causing first gear section 54 to rotate in a clockwise direction on second gear section 55 to articulate end effector 70 with respect to elongate hollow shaft 40 in a right hand direction as shown in FIG. 5.

By fixing first gear section 54 with regard to end effector 70, and fixing second gear section 55 with respect to the elongate hollow shaft 40, a centerline 56 of the articulation segment 50 will be at respective angles 58 and 59 with respect to the elongate hollow shaft 40 and the end effector 70 as best shown in FIG. 2. Both of the respective angles 58 and 59 will be greater than zero when the end effector 70 is articulated in either direction away from the straight configuration shown in FIG. 3. Although each of the gear sections 54 and 55 are shown as having equal radii 57, those skilled in the art will appreciate that different radii could be utilized without departing from the present disclosure. Those skilled in the art will recognize that angles 58 and 59 will be equal when gear sections 54 and 55 have equal radii 57 as shown, but be different when the radii are different.

As best shown in FIG. 5, when end effector 70 is in the form of a clamp 76, a biasing spring 75 may be operably positioned to bias the movable jaw 96 away from fixed jaw 95. Jaws 96 and 95 may be closed by pivoting the pair of handles 30 from their spread configuration 34 to the contracted configuration 33. This may be accomplished by utilizing an effector cable 73 that has one end attached to movable jaw 96 and its opposite end attached to a pull rod 43. The middle section of effector cable 73 extends through fixed jaw 95, through articulation segment 50 and down through elongate hollow shaft 40. Handles 31 and 32 may be coupled to pull rod 43 via struts 45, 46, 47 and 48. A pin 49 may connect the end of the struts 45-48 to the end of pull rod 43 remote from its connection to effector cable 73. In this way, pivotal motion of handles 31 and 32 is transformed into linear motion of pull rod 43 in order to increase tension in effector cable 73 to move end effector 70 from its first configuration toward its second configuration against the action biasing spring 75. Although not necessary, an additional biasing spring may be located between handles 31 and 32 to bias them toward either the contracted configuration 33 or the spread configuration 34 as desired.

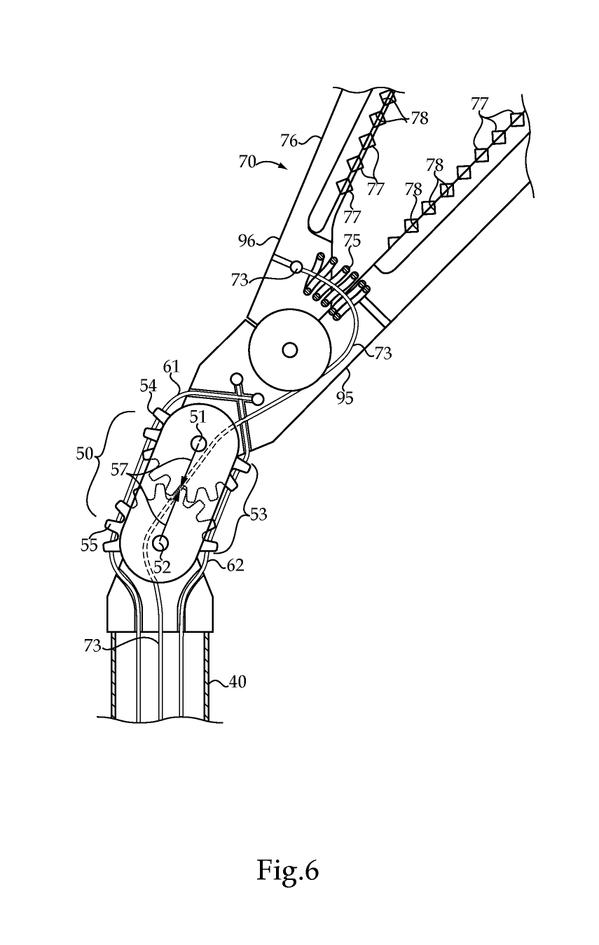

When end effector 70 takes the form of a clamp 76, as shown, the respective fixed jaw 95 and movable jaw 96 may be equipped with gripping features to better enable the device to gain purchase on internal body tissues and/or organs. The fixed jaw 95 and movable jaw 96 may include a plurality of pyramid shaped teeth 77 that are received in pyramid shaped indentations 78 when the clamp 76 is in its second configuration 72. In the illustrated embodiment, both the fixed jaw 95 and the movable jaw 96 include counterpart pyramid shaped teeth 77 and pyramid shaped indentations 78 as best shown in FIG. 6.

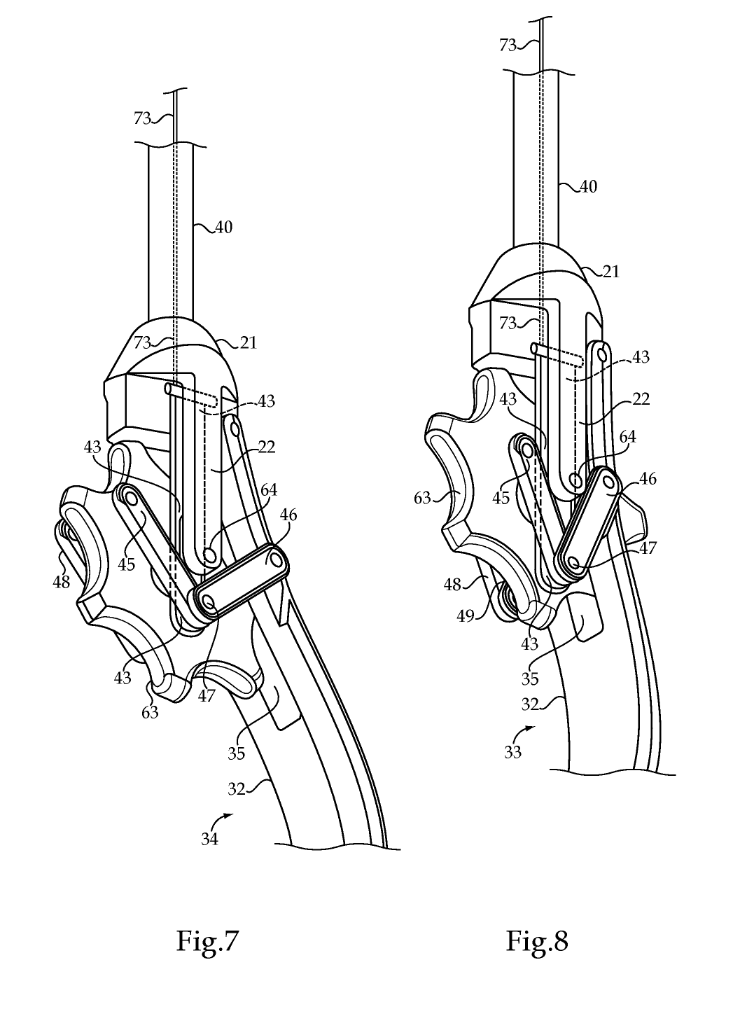

So that surgical hand instrument 20 might be operated by a user with one hand, the steering wheel 63 may be sized and positioned as shown so that the operators thumb could rotate steering wheel 63 simultaneously with movement of handles 31 and 32. This may be accomplished by mounting steering axis 64 between a pair of hub extensions 22, and shaping each of the handles 31 and 32 to define a slot 35 that may receive a portion of steering wheel 63, as best shown in FIGS. 7 and 8. Thus, the steering axis 64 remains fixed with regard to hub 21 regardless of the positioning of the pair of handles 30, and allows for independent operation of the articulation feature separate from opening and closure of the end effector 70.

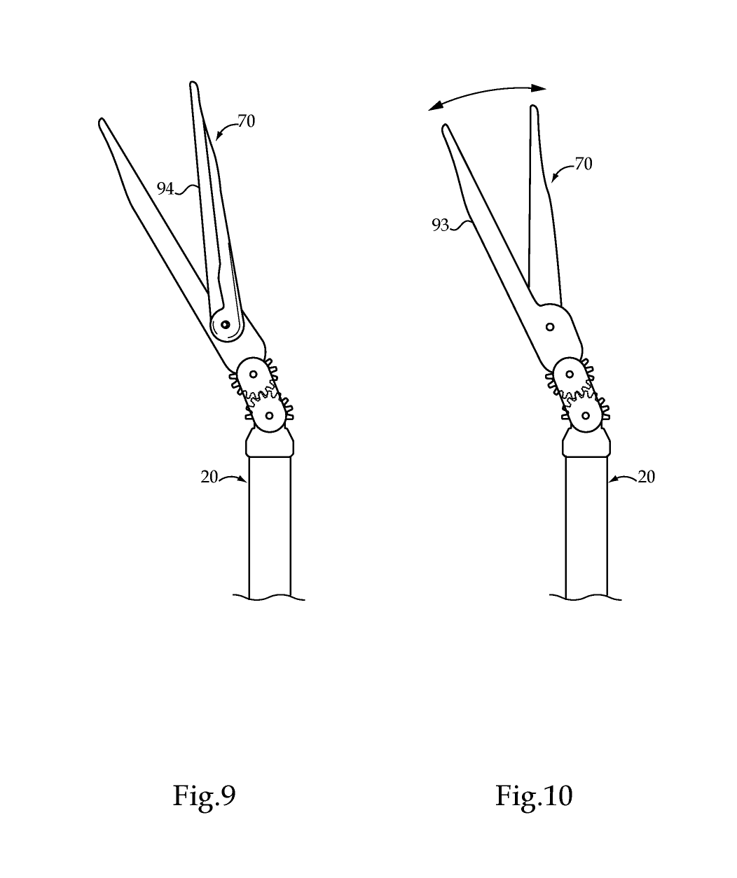

Although surgical hand instrument 20 has been illustrated as including an end effector 70 in the form of a clamp 76, FIGS. 8 and 9 show alternative end effector strategies. FIG. 9 shows an end effector 70 in the form of scissors 94. Those skilled in the art will appreciate that the effector cable 73 for the embodiment shown in FIG. 9 would be much the same as the set up shown for the clamp 76 of the embodiment shown in FIGS. 1-8. FIG. 10 shows an alternative end effector 70 in the form of a spreader 93. In such a case, those skilled in the art will appreciate that the end effector cable would be connected in a reversed configuration so that movement of the pair of handles from the spread configuration 34 toward the contracted configuration 33 would result in the spreader 93 moving toward a first configuration. Reconfiguring end effector cable 73 to facilitate this action should be well within ordinary skill in the art and need not be taught here. In either case, no matter what end effector 70 is chosen, and others apart from scissors, clamps and spreaders would also fall within the intended scope of the present disclosure, all versions would include the articulation feature that utilizes a pair of meshed gears 53 as described earlier.

In all cases, the surgical hand instrument 20 will have a proximal pair of handles 30 with which the user will interact, and a distal end with an end effector 70 that manipulates the patient's tissue. Between the handles 30 and the elongate shaft 40 is a hub 21, off of which the various input features are mounted. These include plier like handles 31 and 32 that are used to actuate the grasper jaws 95 and 96 of clamp 76. The centralized steering wheel 64 is used to actuate the articulation feature to pivot the end effector 70 relative to the centerline 41 of elongate hollow shaft 40. The cables 61, 62, 73 running through the elongate hollow shaft 40 are utilized to transmit forces to the articulation actuator 60 for the articulation segment 50 as well as the end effector 70. The end effector 70 components, and the elongate hollow shaft 40 may be manufactured from a strong, biocompatible metal, or any other material suitable and known in the art. The pair of handles 30 may be manufactured from a strong biocompatible polymer or a biocompatible metal or any other suitable material. The high stress pieces such as axles and fasteners may be made from a suitable metal. As the surgical hand instrument 20 is being assembled, the flexible cable material of effector cable 73, left turn cable 61 and right turn cable 62 may all be made from multi-strand, stainless steel or wire rope that is laced through the device and anchored with proper tension.

### INDUSTRIAL APPLICABILITY

The present disclosure is generally applicable to surgical hand instruments. The surgical hand instrument 20 of the present disclosure is specifically applicable to laparoscopic procedures where the surgeon will need to get access to an internal tissue/organ, manipulate it into a given position and hold the tissue in place while surgery is taking place. Thus, the surgical hand instrument 20 of the present disclosure is specifically applicable to a device that could be passed through another device, such as a trocar 11, to perform any of the wide variety of surgical procedures known in the art and to become known.

In a typical procedure, the surgical hand instrument 20 will be first configured with the end effector 70 in its second configuration 72 as shown in FIG. 3. At this point, the end effector 70 may be moved through a trocar 10 while the end effector 70 has a cross sectional dimension 74 that is equal to or less than an outer diameter 42 of elongate hollow shaft 40. The operator may then rotate surgical hand instrument 20 about a trocar axis 12, which may be coincident with the axis 41 of elongate hollow shaft 40. This may allow the user to orient the articulation axis, which is parallel to the central axes of the meshed gear sections 53. The end effector 70 may then be articulated relative to the elongate hollow shaft 40 responsive to movement of the articulation actuator 60, the left turn cable 61 and the right turn cable 62. Next, the user may move the end effector 70 from the second configuration 72 to the first configuration 71 for grasping an organ or tissue responsive to pivoting the left handle 31 relative to the right handle 32 and moving the effector cable 73. With the tissue or organ thus grasped, the surgical hand instrument 20 may then be repositioned relative to trocar 10 by further movement along trocar axis 11. Before or after this repositioning, the end effector 70 may be moved from the first configuration 71 to the second configuration 72 to grasp the tissue or organ and hold the same in place. Those skilled in the art will appreciate that when the end effector 70 is articulated, the pair of meshed gears section 53 of the articulation segment 50 will rotate relative to each other. If equipped, the position of the end effector 70 may be locked with ratchet 90. The ratchet 90 of the present disclosure may be useful in maintaining a grasping force on the tissue or organ in question. In addition, an external fixturing device (not shown) may be utilized while the user performs other aspects of the surgical procedure. The ratchet 90 may then be unlocked by actuating release 91 and hence the position of end effector 70.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A surgical hand instrument comprising:
a hub;
a pair of handles that includes a left handle and a right handle attached to the hub and being pivotable relative to each other between a spread configuration and a contracted configuration;
an elongate hollow shaft attached to the hub;
an end effector movable between an first configuration and a second configuration;
an articulation segment attached at opposite ends to the elongate hollow shaft and the end effector, respectively;
an articulation actuator positioned between the pair of handles and operably connected to the articulation segment by a left turn cable and a right turn cable;
the end effector being operably coupled to the pair of handles by an effector cable such that the end effector moves between the first and second configurations responsive to the pair of handles moving between the spread and contracted configurations;
the end effector having a cross sectional dimension equal to, or less than, an outer diameter of the elongate hollow shaft in the second configuration, but the cross sectional dimension being greater than the outer diameter of the elongate shaft in the first configuration; and
a pair of meshed gear sections of the articulation segment rotating with respect to each other to articulate the end effector relative to the elongate hollow shaft responsive to movement of the articulation actuator.

2. The surgical hand instrument of claim 1 wherein the left handle and the right handle are located on opposite sides of a plane that contains a centerline of the elongate hollow shaft.

3. The surgical hand instrument of claim 1 wherein the articulation actuator includes a steering wheel rotatable about a steering axis that is intersected by, and perpendicular to, a centerline of the elongate hollow shaft.

4. The surgical hand instrument of claim 1 including a ratchet operably coupled to the pair of handles to prevent the pair of handles from pivoting toward the spread configuration; and
a release operably coupled to disengage the ratchet to permit the pair of handles to pivot toward the spread configuration.

5. The surgical hand instrument of claim 1 including a spring operably positioned to bias the end effector toward the first configuration.

6. The surgical hand instrument of claim 1 wherein the pair of meshed gear sections includes a first gear sections fixed with respect to the elongate hollow shaft, and a second gear section fixed with respect to the end effector.

7. The surgical hand instrument of claim 1 wherein a centerline of the articulation segment is at respective angles greater than zero with respect to the end effector and the elongate hollow shaft when the end effector is articulated with respect to the elongate hollow shaft.

8. The surgical hand instrument of claim 1 wherein the pair of meshed gear sections have equal radii.

9. The surgical hand instrument of claim 1 wherein the end effector is one of a clamp, a spreader and a scissors.

10. The surgical hand instrument of claim 9 wherein the end effector is a clamp with a plurality of pyramid shaped teeth that are received in pyramid shaped indentations when the clamp is in the second configuration.

11. A surgical instrument assembly comprising:
a trocar;
a surgical hand instrument extending through the trocar, and that including:
a hub;
a pair of handles that includes a left handle and a right handle attached to the hub and being pivotable relative to each other between a spread configuration and a contracted configuration;
an elongate hollow shaft attached to the hub;
an end effector movable between an first configuration and a second configuration;
an articulation segment attached at opposite ends to the elongate hollow shaft and the end effector, respectively;
an articulation actuator positioned between the pair of handles and operably connected to the articulation segment by a left turn cable and a right turn cable;
the end effector being operably coupled to the pair of handles by an effector cable such that the end effector moves between the first and second configurations responsive to the pair of handles moving between the spread and contracted configurations; and
a pair of meshed gear sections of the articulation segment rotating with respect to each other to articulate the end effector relative to the elongate hollow shaft responsive to movement of the articulation actuator.

12. The surgical instrument assembly of claim 11 wherein the left handle and the right handle are located on opposite sides of a plane that contains a centerline of the elongate hollow shaft.

13. The surgical instrument assembly of claim 12 wherein the articulation actuator includes a steering wheel rotatable about a steering axis that is intersected by, and perpendicular to, a centerline of the elongate hollow shaft.

14. The surgical instrument assembly of claim 13 wherein a centerline of the articulation segment is at respective angles greater than zero with respect to the end effector and the elongate hollow shaft when the end effector is articulated with respect to the elongate hollow shaft.

15. The surgical instrument assembly of claim 12 wherein the pair of meshed gear sections includes a first gear sections fixed with respect to the elongate hollow shaft, and a second gear section fixed with respect to the end effector.

16. A method of operating a surgical hand instrument that includes a pair of handles that include a left handle and a right handle attached to a hub; an elongate hollow shaft attached to the hub; an articulation segment attached at opposite ends to the elongate hollow shaft and an end effector, respectively; an articulation actuator positioned between the pair of handles and operably connected to the articulation segment by a left turn cable and a right turn cable; the end effector being operably coupled to the pair of handles by an effector cable; and the method comprising the steps of:
moving the end effector through a trocar while the end effector is in a second configuration;
rotating the surgical hand instrument about a trocar axis;
articulating the end effector relative to the elongate hollow shaft responsive to movement of the articulation actuator, the left turn cable and the right turn cable;
moving the end effector from the second configuration to an first configuration responsive to pivoting the left handle relative to the right handle and moving the effector cable;
repositioning the surgical hand instrument relative to the trocar;
moving the end effector from the first configuration toward the second configuration; and
wherein the articulating step includes rotating a pair of meshed gear sections of the articulation segment relative to each other.

17. The method of claim 16 wherein the end effector is a clamp;
the end effector is moved to the second configuration responsive to movement of the pair of handles to a contracted configuration, and the end effector is moved to the first configuration responsive to movement of the pair of handles to a spread configuration; and
biasing the end effector toward the first configuration.

18. The method of claim 17 including a step of locking a position of the end effector with a ratchet positioned between the left handle and the right handle; and
actuating a release to unlock the ratchet and the position of the end effector.

19. The method of claim 16 wherein the articulation actuator includes a steering wheel; and
the articulating step is performed responsive to rotation of the steering wheel.

20. The method of claim 19 wherein the left turn cable is tensioned responsive to rotation of the steering wheel in a counterclockwise direction; and
the right turn cable is tensioned responsive to rotation of the steering wheel in a clockwise direction.
